SEQUENCE LISTING (20 sequences)

SEQ ID NO: 1 - protein, 13 aa, Artificial Sequence
  Synthetic Polypeptide
GPQNQRNAPRITF

SEQ ID NO: 2 - protein, 9 aa, Artificial Sequence
  Synthetic Polypeptide
QRNAPRITF

SEQ ID NO: 3 - protein, 9 aa, Artificial Sequence
  Synthetic Polypeptide
FPRGQGVPI

SEQ ID NO: 4 - protein, 14 aa, Artificial Sequence
  Synthetic Polypeptide
KMKDLSPRWYFYYL

SEQ ID NO: 5 - protein, 11 aa, Artificial Sequence
  Synthetic Polypeptide
GQQQQGQTVTK

SEQ ID NO: 6 - protein, 12 aa, Artificial Sequence
  Synthetic Polypeptide
GQQQQGQTVTKK

SEQ ID NO: 7 - protein, 13 aa, Artificial Sequence
  Synthetic Polypeptide
RTATKAYNVTQAF

SEQ ID NO: 8 - protein, 15 aa, Artificial Sequence
  Synthetic Polypeptide
RTATKAYNVTQAFGR

SEQ ID NO: 9 - protein, 11 aa, Artificial Sequence
  Synthetic Polypeptide
ATKAYNVTQAF

SEQ ID NO: 10 - protein, 13 aa, Artificial Sequence
  Synthetic Polypeptide
ATKAYNVTQAFGR

SEQ ID NO: 11 - protein, 9 aa, Artificial Sequence
  Synthetic Polypeptide
KAYNVTQAF

SEQ ID NO: 12 - protein, 11 aa, Artificial Sequence
  Synthetic Polypeptide
KAYNVTQAFGR

SEQ ID NO: 13 - protein, 15 aa, Artificial Sequence
  Synthetic Polypeptide
AQFAPSASAFFGMSR

SEQ ID NO: 14 - protein, 15 aa, Artificial Sequence
  Synthetic Polypeptide
NFKDQVILLNKHIDA

SEQ ID NO: 15 - protein, 9 aa, Artificial Sequence
  Synthetic Polypeptide
KTFPPTEPK

SEQ ID NO: 16 - protein, 10 aa, Artificial Sequence
  Synthetic Polypeptide
KTFPPTEPKK

SEQ ID NO: 17 - protein, 14 aa, Artificial Sequence
  Synthetic Polypeptide
KKADETQALPQRQK

SEQ ID NO: 18 - protein, 13 aa, Artificial Sequence
  Synthetic Polypeptide
KADETQALPQRQK

SEQ ID NO: 19 - protein, 10 aa, Artificial Sequence
  Synthetic Polypeptide
GPQNQRNAPR

SEQ ID NO: 20 - protein, 13 aa, Artificial Sequence
  Synthetic Polypeptide
LQLPQGTTLPKGF

(19) United States
(12) Patent Application Publication
(10) Pub. No.: US 2023/0326544 A1
(43) Pub. Date: Oct. 12, 2023

(54) Computerized Tool For Prediction of Proteasomal Cleavage

(71) Applicant: IMMUNITYBIO, INC., Culver City, CA (US)

(72) Inventors: Jeremi SUDOL, Culver City, CA (US); Kamil A. WNUK, Culver City, CA (US); Andrew NGUYEN, Culver City, CA (US); John Zachary SANBORN, Culver City, CA (US)

(21) Appl. No.: 18/023,190

(22) PCT Filed: Aug. 25, 2021
(86) PCT No.: PCT/IB2021/057770

§ 371 Date: Feb. 24, 2023

Related U.S. Application Data
(60) Provisional application No. 63/072,083, filed on Aug. 28, 2020.

(51) Int. Cl. G16B 15/30; G16B 40/20

(52) U.S. Cl. CPC G16B 15/30; G16B 40/20

(57) ABSTRACT

A method of preparing a vaccine includes providing an immune epitope database; providing a neural network; receiving data corresponding to at least one protein into the neural network; receiving data corresponding to one or more candidate peptides corresponding to potential cleavage products of the at least one protein, or determining, using the neural network, data corresponding to one or more candidate peptides corresponding to potential cleavage products of the at least one protein; calculating, using the neural network, a probability of cleavage of the protein to result in each of the one or more candidate peptides; and outputting a signal corresponding to the calculated probability. An architecture having two channel output, i.e., output of a C-terminal cleavage and an N-terminal cleavage, is described. Related devices, apparatuses, systems, techniques, articles and non-transitory computer-readable storage medium are also described.

### CROSS-REFERENCE TO RELATED APPLICATION

This application is a PCT International Application and claims the benefit and priority of U.S. Provisional Application No. 63/072,083, filed Aug. 28, 2020. The entire disclosure of the above application is incorporated herein by reference.

### REFERENCE TO A SEQUENCE LISTING

The sequence listing entitled “62887626_1.txt”, created on Aug. 20, 2020 and 8,192 bytes in size, is hereby incorporated by reference.

### TECHNICAL FIELD

The present disclosure relates to a device, system and method for analysis of proteasomal cleavage of a protein. Specifically, the present disclosure relates to an architecture, devices, systems and related methods for analyzing cleavage of a given protein, for example, in FASTA format, and for outputting possible resulting peptides in a length range above a confidence threshold including high probability cleavage sites, and the like.

### BACKGROUND

Major histocompatibility complex (MHC), including MHC Class I (MHC-I) and MHC Class II (MHC-II), are important to the immune system's ability to distinguish “self” antigens from pathogen. For example, MHC-I is expressed on nearly every cell in the body, and it is responsible for presenting self- and foreign-derived display peptides on the cell surface to lymphocytes. Display peptide presentation by MHC-I is one of the first steps of an adaptive immune response toward destruction of diseased cells or for preservation of healthy cells. See, e.g., Neefjes, Jacques, et al., Nature Reviews Immunology 11.12 (2011): 823-836; Mester et al., Cellular and Molecular Life Sciences 68.9 (2011): 1521-1532.

Tumor antigen recognition by the immune system is of increasing interest for the purposes of cancer and other disease treatments. See, e.g., Kalaora, Shelly, et al., Nature Communications 11.1 (2020): 1-12. For example, cancer vaccines use tumor-associated antigens or neoantigens to prime a patient's immune system to target the tumor. However, identifying antigens that may be useful for this purpose is difficult. It is even more challenging to identify candidate antigens for personalized treatment of a particular patient.

The present inventors developed improvements in devices and methods for analysis of protein cleavage that overcome at least the above-referenced problems with the devices and methods of the related art.

### SUMMARY

A method of preparing a vaccine is provided. The method may include a peptide antigen or an immunotherapy treatment for cancer comprising a peptide antigen. A device may be provided. The device may have at least one processor and a memory storing at least one program for execution by the at least one processor. The at least one program may include instructions, which, when executed by the at least one processor cause the at least one processor to perform operations. The operations may include providing an immune epitope database. The operations may include providing a neural network. The operations may include receiving data corresponding to at least one protein into the neural network. The operations may include receiving data corresponding to one or more candidate peptides corresponding to potential cleavage products of the at least one protein, or determining, using the neural network, data corresponding to one or more candidate peptides corresponding to potential cleavage products of the at least one protein. The operations may include calculating, using the neural network, a probability of cleavage of the protein to result in each of the one or more candidate peptides. The operations may include outputting a signal corresponding to the calculated probability.

A system for preparing a vaccine is provided. The system may include a peptide antigen or an immunotherapy treatment for cancer comprising a peptide antigen. The system may include a device having at least one processor and a memory storing at least one program for execution by the at least one processor. The at least one program may include instructions, when, executed by the at least one processor cause the at least one processor to perform operations. The operations may include providing an immune epitope database. The operations may include providing a neural network. The operations may include receiving data corresponding to at least one protein into the neural network. The operations may include receiving data corresponding to one or more candidate peptides corresponding to potential cleavage products of the at least one protein, or determining, using the neural network, data corresponding to one or more candidate peptides corresponding to potential cleavage products of the at least one protein. The operations may include calculating, using the neural network, a probability of cleavage of the protein to result in each of the one or more candidate peptides. The operations may include outputting a signal corresponding to the calculated probability.

A non-transitory computer-readable storage medium storing at least one program for preparing a vaccine. The vaccine may include a peptide antigen or an immunotherapy treatment for cancer including a peptide antigen. The at least one program may be for execution by at least one processor and a memory storing the at least one program. The at least one program may include instructions, when, executed by the at least one processor cause the at least one processor to perform operations. The operations may include providing an immune epitope database. The operations may include providing a neural network. The operations may include receiving data corresponding to at least one protein into the neural network. The operations may include receiving data corresponding to one or more candidate peptides corresponding to potential cleavage products of the at least one protein, or determining, using the neural network, data corresponding to one or more candidate peptides corresponding to potential cleavage products of the at least one protein. The operations may include calculating, using the neural network, a probability of cleavage of the protein to result in each of the one or more candidate peptides. The operations may include outputting a signal corresponding to the calculated probability.

Each of the method, the system and the non-transitory computer-readable storage medium may include one or more of the following features in any suitable combination.

The method, the system, and/or the non-transitory computer-readable storage medium may further include choosing a peptide antigen based on the signal corresponding to the calculated probability and preparing the vaccine with the chosen peptide antigen. The choosing the peptide antigen based on the signal corresponding to the calculated probability may be based on a determination of whether the calculated probability is within a predetermined range of values.

The calculating, using the neural network, the probability of cleavage for each of the one or more candidate peptides may include: calculating, using the neural network, a probability of cleavage for at least one N-terminal of each of the one or more candidate peptides; or calculating, using the neural network, a probability of cleavage for at least one C-terminal of each of the one or more candidate peptides.

The calculating, using the neural network, the probability of cleavage for each of the one or more candidate peptides may include: calculating, using the neural network, a probability of cleavage for at least one N-terminal of each of the one or more candidate peptides; and independent of the N-terminal calculation, calculating, using the neural network, a probability of cleavage for at least one C-terminal of each of the one or more candidate peptides.

The operations may further include: determining, using the neural network, data corresponding to one or more neighboring variants of the one or more candidate peptides; and calculating a probability of cleavage for the one or more neighboring variants.

The immune epitope database may include data representing one or more unique antigen proteins, one or more unique peptides, one or more unique peptide/protein pairs, and one or more decoys.

The immune epitope database may be restricted to major histocompatibility complex (WIC) pathways.

The immune epitope database may be restricted to WIC Class I (WIC-I) pathways.

The immune epitope database may be restricted to human-only immune epitopes.

The immune epitope database may be restricted to sequences that positively bind to WIC.

The immune epitope database may include tandem mass spectrometry data where a single WIC-allele is not identified.

A flank size for each of the one or more candidate peptides may be greater than or equal to 6 and less than or equal to 20.

A flank size for each of the one or more candidate peptides may be 12.

A measurement of an accuracy of the calculating, using the neural network, the probability of cleavage for each of the one or more candidate peptides may include a receiver operating characteristic (ROC), and where an ROC closest to 1.0 is ideal.

The neural network may include: one or more convolutional layers; and one or more fully connected layers. The one or more convolutional layers may consist of a single convolutional layer.

The neural network may include: one or more convolutional layers; and one or more fully connected layers. The one or more convolutional layers may comprises the one or more convolutional layers in parallel. Each of the one or more convolutional layers may have a different size kernel.

One or more candidate peptides may be modeled without an explicit encoding of a cleavage marker.

The neural network may include a parametric rectified linear unit activation function.

The outputting the signal corresponding to the calculated probability may include one or more of the following: generation of a first table including a position column, an antigen marker, a probability of cleavage, and an indicator of cleavage or a pad; generation of a second table including data for an N-terminal and data for a C-terminal, where each of the data for the N-terminal and the data for the C-terminal includes: a position column, an antigen marker, an N-terminal probability of cleavage, an N-terminal indicator of cleavage or a pad; a C-terminal probability of cleavage, and a C-terminal indicator of cleavage or a pad; generation of a third table including a candidate peptide column, a length column, an N-terminal probability, and a C-terminal probability; and generation of a fourth table including a candidate peptide column, an N-terminal probability, and a C-terminal probability, where the candidate peptide column includes one or more neighboring variants of the one or more candidate peptides.

The vaccine may be for an infectious disease.

The vaccine may be for a cancer.

The at least one protein may be a tumor-associated antigen.

The at least one protein may be a neoantigen.

The at least one protein may be an antigen from a virus, bacterium, fungus, protozoa, prion, or helminth.

The outputting may include two channel output. The two channel output may include output of a probability of a C-terminal cleavage and a probability of an N-terminal cleavage.

These and other capabilities of the disclosed subject matter will be more fully understood after a review of the following figures, detailed description, and claims.

### DESCRIPTION OF DRAWINGS

These and other features will be more readily understood from the following detailed description taken in conjunction with the accompanying drawings, in which:

FIG. 1 is a diagram of an architecture according to an exemplary embodiment;

FIG. 2 is a plot of performance on withheld validation data of a benchmark versus a plain update versus the plain update with batch normalization according to an exemplary embodiment;

FIG. 3A is a plot of performance of a single conversion layer according to an exemplary embodiment;

FIG. 3B is a diagram of an architecture of the single convolutional layer of FIG. 3A according to an exemplary embodiment;

FIG. 4A is a plot of performance of concatenated multiple convolutional layers according to an exemplary embodiment;

FIG. 4B is a diagram of an architecture of the concatenated multiple convolutional layers of FIG. 4A according to an exemplary embodiment;

FIG. 5 is a plot of performance of L2 regularization according to an exemplary embodiment;

FIG. 6 is a plot of performance of variants of convolutional layers according to an exemplary embodiment;

FIG. 7A is a plot of performance of a first encoded variant versus benchmark according to an exemplary embodiment;

FIG. 7B is a plot of performance of a second encoded variant versus benchmark according to an exemplary embodiment;

FIG. 7C is a plot of performance of a third encoded variant versus benchmark according to an exemplary embodiment;

FIG. 8A is a plot of a rectified linear unit (ReLU) activation function according to an exemplary embodiment;

FIG. 8B is a plot of a Leaky ReLU activation function according to an exemplary embodiment;

FIG. 8C is a plot of a parametric ReLU (PReLU) activation function according to an exemplary embodiment;

FIG. 9 is a plot of performance of a PReLU model versus two previous top performing models according to an exemplary embodiment;

FIG. 10A is a plot of performance of using C-terminal, N-terminal, or a combination of the N-terminal and the C-terminal according to an exemplary embodiment;

FIG. 10B is a diagram of an architecture of the C-terminal, the N-terminal and the combination of the N-terminal and the C-terminal of FIG. 10A according to an exemplary embodiment;

FIG. 11 is a flow chart of a method for protein cleavage analysis according to an exemplary embodiment; and

FIG. 12 is a schematic diagram of a computer device or system including at least one processor and a memory storing at least one program for execution by the at least one processor according to an exemplary embodiment.

It is noted that the drawings are not necessarily to scale. The drawings are intended to depict only typical aspects of the subject matter disclosed herein, and therefore should not be considered as limiting the scope of the disclosure. Those skilled in the art will understand that the structures, systems, devices, and methods specifically described herein and illustrated in the accompanying drawings are non-limiting exemplary embodiments and that the scope of the present invention is defined solely by the claims.

### DETAILED DESCRIPTION

Diseases such as cancer may be associated with abnormalities (e.g., genetic mutations) that are unique to the patient, or to a subset of patients. Such differences allow for customization of treatment (personalized medicine) to that patient. The present device, system and method are useful to providing a customized vaccine or treatment for each patient, e.g., a bespoke customized cancer therapy. That is, the vaccine or treatment may be customized for a patient's particular antigen expression associated with the disease, e.g., cancer. For example, target cancer-associated antigens or neoantigens may be chosen to be included into the vaccine or treatment. The present device, system and method inform the choice of antigen selection. The present device, system and method may indicate peptides resulting from intracellular cleavage of a protein and presentation of the resulting peptide(s) on a surface of an antigen presenting cell. The present device, system and method may identify one or more peptides that are likely to emerge from proteasomal cleavage within a cell. The present device, system and method may select target antigens for inclusion in a vaccine, e.g., a cancer vaccine for cancer therapy. The present device, system and method may select target antigens for inclusion in bespoke customized cancer therapy or disease therapy.

The present device, system and method may facilitate development of generic vaccines for viruses. The present device, system and method may facilitate development of a vaccine. The present device, system and method may facilitate development of a treatment for infection by a pathogen, e.g., infection by a virus, bacteria, and the like. The present device, system and method may facilitate development of a treatment for coronavirus disease 2019 (COVID-19), an infectious disease caused by severe acute respiratory syndrome coronavirus 2 (SARS-CoV-2).

Given at least one protein, the present device, system and method is configured to output at least one peptide in a length range above a confidence threshold. Also, given at least one candidate peptide and a protein, the present device, system and method is configured to output a probability of cleavage. Further, the present device, system and method may output at least one neighboring variant with an associated probability of cleavage.

Still further, given at least one protein and/or defective ribosomal product (DRiP), the present device, system and method is configured to output one or more peptides that are expected to result from cleavage of the protein or DRiP by one or more of the proteasome, a protease, ER-associated aminopeptidase associated with Ag processing (ERAAP), transporter associated with Ag-processing (TAP), the cytosolic Ag-processing (CAP) pathway, or any other antigen processing or proteolytic pathway in a cell, in particular an antigen-presenting cell.

The proteasome may include constitutive-proteasome. The proteasome may include an immunoproteasome. The proteasome may include proteasomal cleavage specificity. The proteasome may include proteasomal variants. The proteasome may include aminopeptidases. The proteasome may include proteasome-based peptide splicing. The proteasome may include non-linear epitopes.

The TAP may include binding specificity. The TAP may include TAP-pathway disruption or evasion. The TAP may include TAP-independent processing. The TAP may include endosomal recycling.

In some exemplary embodiments, a model may include the proteasome including constitutive-proteasome, an immunoproteasome, proteasomal cleavage specificity, proteasomal variants and aminopeptidases; the model may include the TAP including binding specificity; and the model may include the MHC-I including binding specificity.

As the input, a database may be utilized. In some embodiments, the database may include antigens or peptides that result from cleavage by a cell. In some embodiments, the antigens or peptides are presented on the surface of an antigen-presenting cell. In some embodiments, the antigens or peptides are presented by MHC. In some embodiments, the MHC is MHC-I. In some embodiments, the MHC is MHC-II. In some exemplary embodiments, the Immune Epitope Database (IEDB) 2020 may be utilized (e.g., “mhc_full_ligands”, dated Feb. 25, 2020) (see, also, Vita, Randi, et al., “The immune epitope database (IEDB): 2018 update”, Nucleic Acids Research 47.D1 (2019): D339-D343). In one exemplary embodiment, the IEDB 2020 includes about 81,533 unique antigen proteins, about 285,301 unique peptides, about 438,403 unique peptide/protein pairs, about 4,694,344 decoys (about 10:1), and about 5,000,000 total sequences. The IEDB 2020 includes about 60 times more data than that provided by previous databases or methods, which have on the order of less than about 10,000 ligands. Specifically, the IEDB 2020 utilizes improved data relative to the SYFPEITHI database (Rammensee, H-G., et al., “SYFPEITHI: database for MHC ligands and peptide motifs”, Immunogenetics 50.3-4 (1999): 213-219) and the AntiJen database (Blythe, Martin J., Irini A. Doytchinova, and Darren R. Flower, “JenPep: a database of quantitative functional peptide data for immunology”, Bioinformatics 18.3 (2002): 434-439).

The present device, system and method may receive as input the database, e.g., IEDB 2020, which contains information about peptides that bind (“binding peptides”) or do not bind (non-bionMHC. The database, e.g., IEDB 2020 may or may not include corresponding alleles. The present device, system and method may use information from the database, e.g., IEDB 2020, to inform a predictor as to whether or not a specific location in a protein will be a cleavage site.

The IEDB 2020 includes peptide-protein pairs, and/or that a peptide coming from a particular protein has presented on an MHC molecule. The present device, system and method may incorporate data from the IEDB 2020 as positive examples, i.e., instances where cleavage happened in a particular situation. IEDB 2020 does not provide negative examples. The present device, system and method may include decoys, e.g., peptides not shown to be produced by cleavage of the protein. The decoys may be created by sampling regions within the proteins. Once a protein has been cleaved, within the resulting peptide, there are no additional cleavages. Decoys for a given peptide, i.e., where there are no known cleavages, may be used as a potential negative example. For instance, there may be more than 10 times as many examples as the positive examples because the number of possibilities of negative cleavages.

For example, given a hypothetical protein called “ABCDE”, there may be a first cleavage site between “A” and “B” and a second cleavage site between “D” and “E”, e.g., this may be expressed as “A|BCD|E”, and a positive example for the hypothetical ABCDE protein would be “+BCD”. The present device, system and method may encode the hypothetical ABCDE protein to indicate two cleavage sites and a consideration for sizes of flanks. For example, a flank window may be two (e.g., “Size of flank window=2”), which means, with this setting, the present device, system and method searches for two amino acids to the left of the cleavage site and two amino acids to the right of the cleavage site. So N flank may be expressed as, e.g., “N-flank: .A|BC”, i.e., an empty A, a cleavage site and then BC. This may be an N flank positive example. Similarly, a C-flank may be expressed as, e.g., “C-flank: CD|E.”, which corresponds with C, D, a cleavage site, E and a pad, where, the pad=“.”. Negatives may be expressed as, e.g., “Negatives: AB|CD”, i.e., A, then B, a cleavage site between B and C, and D. That is, this is a negative site, because there was no actual cleavage between B and C for the hypothetical ABCDE protein. “BC|DE” is another example of a negative for this example. This syntax may be used for longer peptides or peptides having numerous examples, positives, negatives, and the like.

For use with the present device, system and method, the database, e.g., IEDB 2020, may be restricted as follows: MHC-I, human-only; only positive (binder) sequences (e.g., ['Positive-High', ‘Positive-Intermediate’, ‘Positive’], quant<=500 NmL); valid epitope source protein; and including tandem mass spectrometry (MS/MS) data where a single MHC-allele is not identified. That is, because the database contains other organisms, i.e., other species besides human, e.g., bovine, mouse, and the like, the database may be restricted to data for humans. The database may be restricted to MHC-I, because MHC-II is a different human category. In some embodiments, the database may include peptides that bind MHC-I and/or MHC-II. The database may be restricted to positive binder sequences (peptides that bind MHC) in order to ensure that the positive examples actually present on the surface of a cell. The database may be restricted to MS/MS data where at least one MHC allele is identified to facilitate MHC binding prediction.

The present device, system and method does not necessarily require input for an allele type. The present device, system and method may produce a generic cleavage prediction for peptides that will be bound to one or more MHC alleles.

The present device, system and method may include a data format. The data format may be, for example, “XXXXX|XXXXX”, where “|” denotes a cleavage site, which may correspond with a center, and which may or may not be explicitly coded, i.e., “XXXXX|XXXXX” may instead be coded as “”XXXXXXXXXX″ with an understanding that the cleavage site is always in the middle, which may be useful for coding with BLOcks SUbstitution Matrix (BLOSUM) encoding or one-hot encoding. The data format may include a symmetric sized flank, i.e., a left side flank symmetric with a right side flank. The flank size may be a parameter.

Padding may be provided, as needed. The padding may be denoted with “.”, e.g., “ . . . XX|XXXXX” . For example, if a cleavage site is at a very extreme part of a protein, either near a C-terminus or N-terminus, if characters are missing for that flank window, then the period (“.”) or any other abstract character may be provided to indicate nothing present.

The flank size may be left as an open parameter in order to determine whether different flank sizes impact the analysis. Through experimentation, a point of diminishing returns was observed where, with a bigger flank size, the amount of useful return was minimized. Since higher flank sizes can undesirably tax compute power, a flank size of about 7 to about 9 was found to be useful in many scenarios.

For each peptide, an n_flank (flank at the N terminus of the peptide), decoys, and a c_flank (flank at the C terminus of the peptide) may be provided. Decoys may be sampled from within a peptide region. The data format may accommodate, for example, 5 folds in a 90/10 train/validation split. That is, cross validation is a process where a given dataset is limited to certain selected parts. During testing, the data does not include data used for training.

Samples may be grouped, for example, by protein. For example, peptides coming from the same protein may be provided in a same dataset, to avoid peptides from the same protein for both the training and the testing.

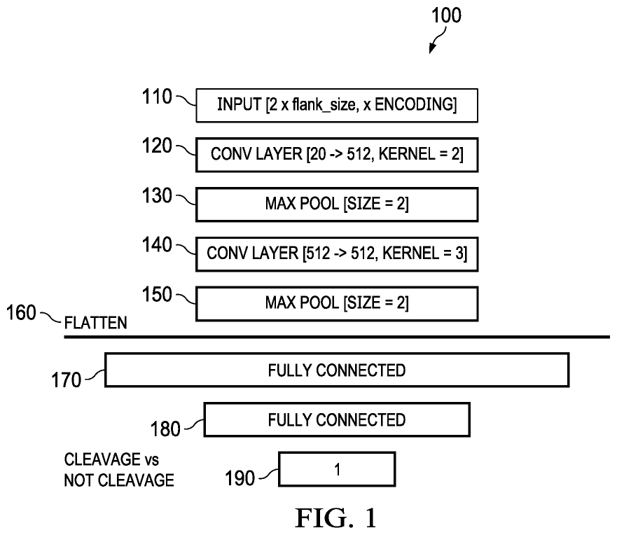

FIG. 1 is a diagram of an architecture 100 according to an exemplary embodiment. The architecture 100 may form the basis of a neural network. The architecture 100 may include a plurality of layers. The plurality of layers may include one or more of an input layer 110, a first convolutional layer 120, a first maximum pooling layer 130, a second convolutional layer 140, a second maximum pooling layer 150, a flattening layer 160, a first fully connected layer 170, a second fully connected layer 180, and a result layer 190.

The input layer 110 may include an encoding such as the above-referenced “XXXXX|XXXXX” (with or without the cleavage symbol, “|”). With the non-limiting example of “XXXXX|XXXXX”, the flank size is 5, and the encoding mechanism may be any one of a suitable type of numerical encoding.

Examples of numerical encoding include one-hot, BLOSUM, nonlinear Fisher (NLF) transformation, and the like. With one-hot, an amino acid is encoded with a number of zeros and a one for every amino acid in a lookup table of 20 amino acids. Also, for example, BLOSUM62 is a substitution matrix that specifies a similarity of one amino acid to another by a score, which reflects a frequency of substitutions found from studying a protein sequence in large databases of related proteins. The number “62” refers to a percentage identity at which sequences are clustered. Encoding a peptide with BLOSUM62 provides a column from a BLOSUM matrix corresponding to an amino acid at each position of a sequence, which produces a 21×9 matrix.

The encoded data may be evaluated with a number of neural network operations. The neural network operations may include convolutional layers, i.e., a convolutional filter may be applied across a sequence, the layer may be activated, and the sequence may respond as activations and or non-activations. A kernel size may determine a size of a filter. The arrows in layers 120 and 140 denote a number of filters, here 20, which is one for each of the encodings, and 512 channels. The analysis may move into a different computational space. The convolutional layers may include pooling (e.g., layer 130), which is a process of taking localized averages of windows to manage a relatively large amount of data being processed and avoid crashing the system. For example, with pooling, within a relatively small window of two, maximum values may be determined and an averaging summarization may be generated, which is inputted into a next layer. In layer 140, the input may have 512 channels, the output may also have 512 channels, and the kernel may have a size of three. The results may be pooled again (e.g., in layer 150). An operation to flatten the entire structure may be performed (e.g., 160), which reduces, for example, 512 separate channels into one, which may be inputted into fully connected layers (e.g., layers 170 and 180). The one or more fully connected layers 170 and 180 may be into a single result layer or single node, e.g., 190. In some exemplary embodiments, the result layer or single node 190 may have a value, and if the value is below 0.5, the value indicates no cleavage, and if the value is above 0.5, then the value indicates a cleavage site. With a value of exactly 0.5, one default may be non-cleavage. Please note, as a practical matter, values near 0.5 would tend not to favor presence of a cleavage site; thus, the value is, at best, ambiguous.

The architecture 100 may include one or more parameters including convolutional layers, a number of convolutional layers, a number of filters, kernel sizes, whether and how to use pooling, a number of fully connected layers, a size of fully connected layers, a number of outputs (e.g., including a two output network to separate an N-terminal or an N-cleavage site and a C-terminal or a C-cleavage site), and different kinds of encodings (e.g., one-hot, BLOSUM, etc.). The architecture 100 may include hyper parameters including a learning rate, adjustments to the learning process, and a batch size. The batch size refers to how many samples are being processed in a smaller window of time, which may impact a convergence of a given network. The data may be divided into subsets for relatively faster initial estimation and exploration of a given parameter space.

In one exemplary embodiment of the architecture 100, flank size was varied, and the results were recorded in terms of receiver operating characteristic (ROC), average precision (PR AUC), and precision (PPV). The initial results are presented in Table 1, as follows:

TABLE 1

| | | | |
|---|---|---|---|
| Flank size 6: | ROC 0.8629 | PR AUC 0.6578 | PPV 0.8606 |
| Flank size 10: | ROC 0.8925 | PR AUC 0.7146 | PPV 0.8732 |
| Flank size 12: | ROC 0.8946 | PR AUC 0.7202 | PPV 0.8748 |
| Flank size 16: | ROC 0.8951 | PR AUC 0.7215 | PPV 0.8756 |
| Flank size 20: | ROC 0.8965 | PR AUC 0.7252 | PPV 0.8765 |

Improvements were observed as a flank size went up to size 12. After the flank size increased beyond 12, limited improvements were observed. Running the system with a flank size of 20 required almost twice as much memory to run as flank size 12. Explorations with a fixed flank size of 12 were performed to obtain best parameters and then the impact of flank size was reevaluated later in the process.

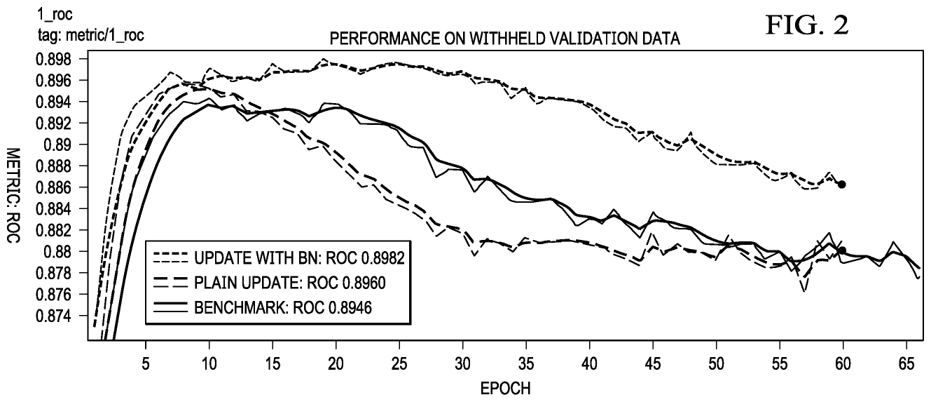

In each of FIGS. 2, 3A, 4A, 5, 6, 7A, 7B, 7C, 9 and 10A, the x-axis denotes epoch, where each epoch is a full run through an entire set of data, and the y-axis denotes the ROC.

FIG. 2 is a plot of performance on withheld validation data of a benchmark versus a plain update versus the plain update with batch normalization according to an exemplary embodiment. FIG. 2 illustrates the results of three different models. The benchmark achieved an ROC of 0.8946. Any spot along the curve may be selected as a single model that may be used going forward with subsequent testing. Typically, one may select a peak of each curve and specify a characteristic or a metric for a given model that can be deployed in a real world scenario. After running the benchmark, an update was made (i.e., e.g., adjustments in a number of filters at each level and variations in filter sizes), and an ROC of 0.8960 was achieved. Batch normalization on the fully connected layers achieved an ROC of 0.8982.

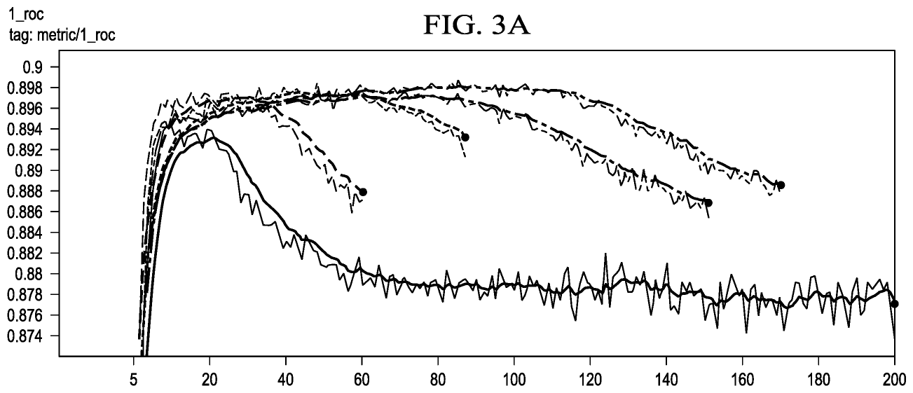

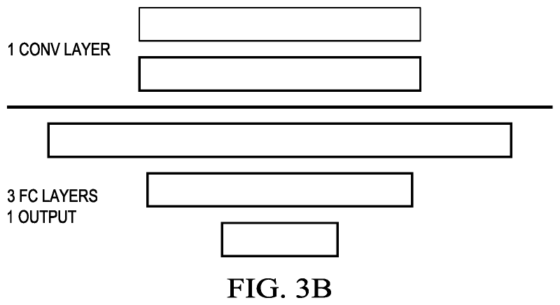

FIG. 3A is a plot of performance of a single conversion layer according to an exemplary embodiment. Three iterations were run versus the previous benchmark established in FIG. 2, and batch normalization was again performed on the fully connected layers. Modest improvement in ROC was observed with the single conversion layer. FIG. 3B is a diagram of an architecture of the single convolutional layer of FIG. 3A according to an exemplary embodiment.

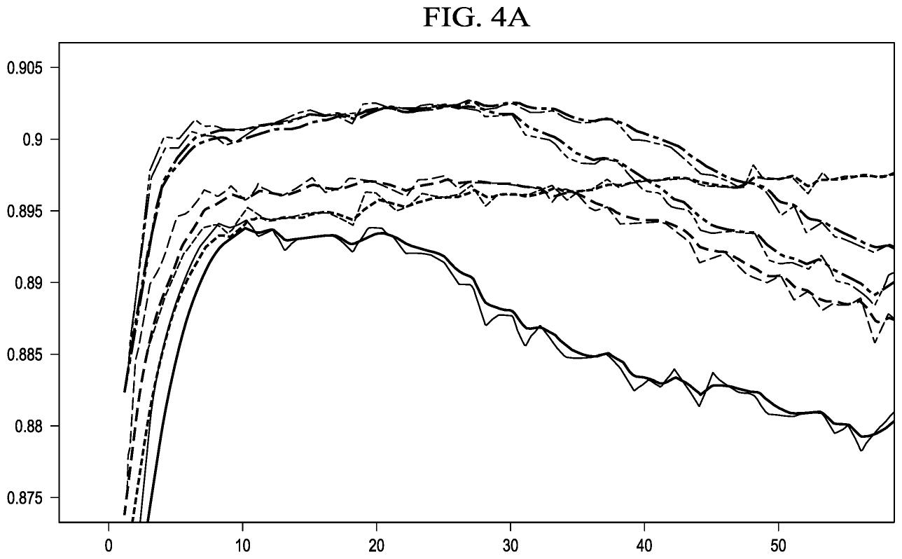

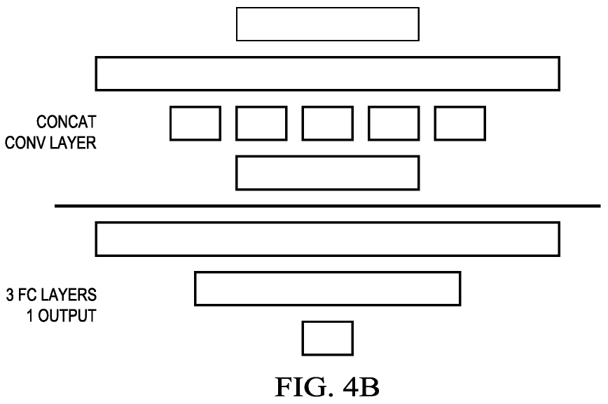

FIG. 4A is a plot of performance of concatenated multiple convolutional layers according to an exemplary embodiment. FIG. 4A represents two or more convolutional kernels in parallel, which are combined into a final layer before being flattened and proceeding with fully connected layers. Here, a kernel size was varied (e.g., one, two, three, five, seven, thirteen), processed in parallel and combined for final processing. A significant impact was observed, i.e., an ROC AUC of 0.9030 was achieved. FIG. 4B is a diagram of an architecture of the concatenated multiple convolutional layers of FIG. 4A according to an exemplary embodiment.

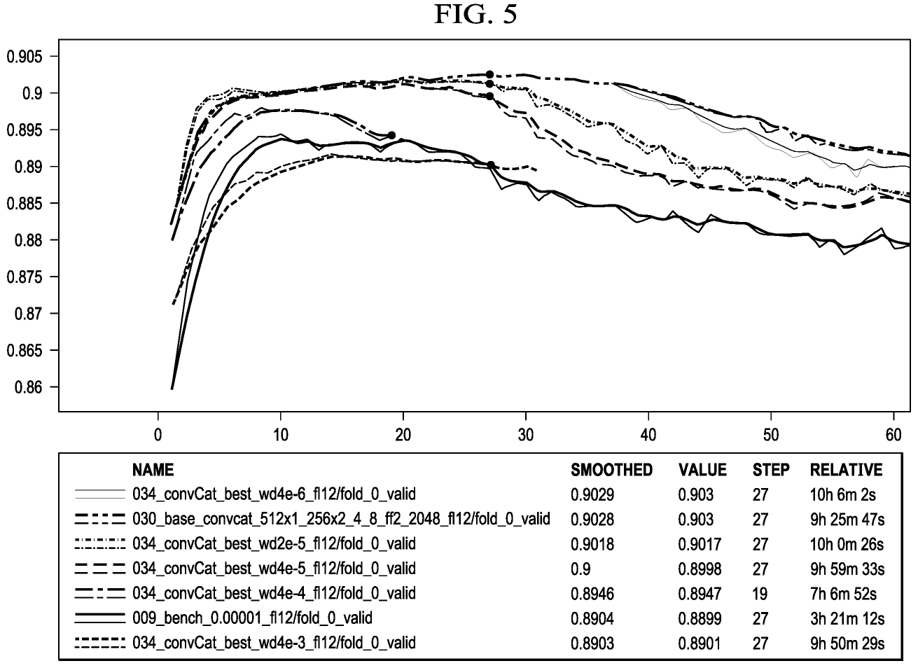

FIG. 5 is a plot of performance of L2 regularization according to an exemplary embodiment. Various types of L2 regularization did not achieve increased performance.

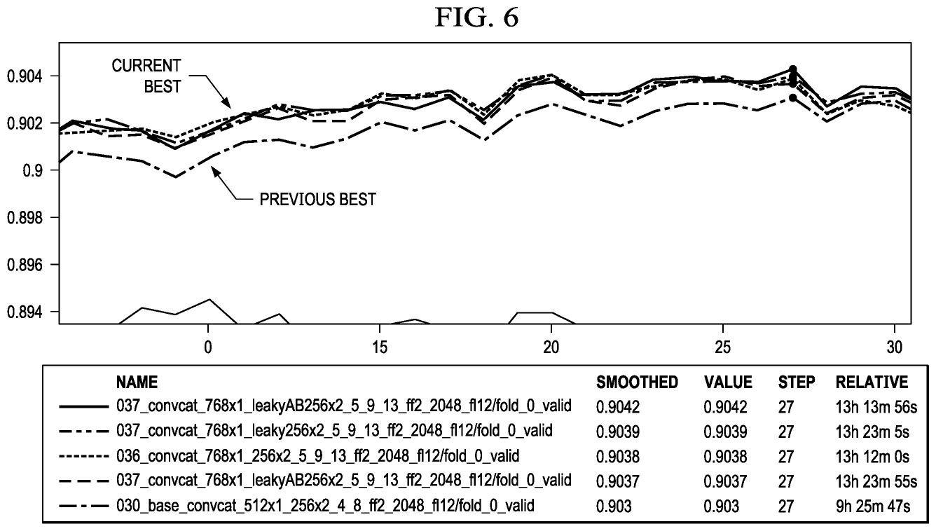

FIG. 6 is a plot of performance of variants of convolutional layers according to an exemplary embodiment. Modest improvement in performance was observed using the variants of the convolutional layers. The variants may include one or more of the following: changing a number of filters; addition of a leaky rectified linear unit (ReLU) (instead of a normal ReLU) for a multi-layer section of the network; addition of a leaky ReLU (instead of the normal ReLU) for a first convolutional layer and the multi-layer section of the network; and addition of the leaky ReLU (instead of the normal ReLU) for the first convolutional layer, the multi-layer section of the network, and a final concatenated fully connected section of the network.

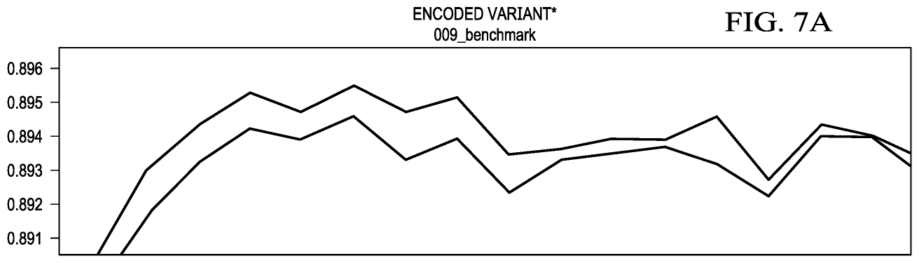

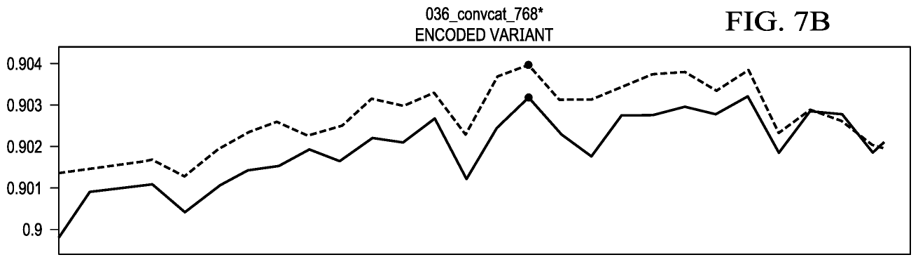

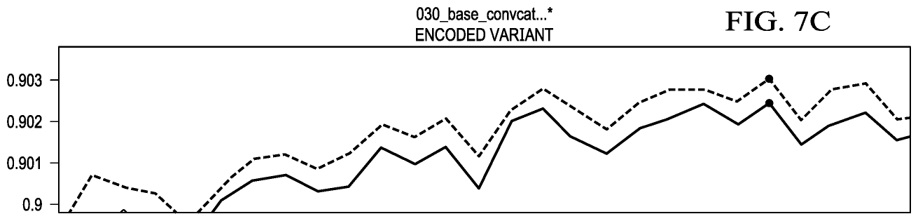

FIG. 7A is a plot of performance of a first encoded variant versus benchmark according to an exemplary embodiment. FIG. 7B is a plot of performance of a second encoded variant versus benchmark according to an exemplary embodiment. FIG. 7C is a plot of performance of a third encoded variant versus benchmark according to an exemplary embodiment. Although an encoded cleavage marker was associated with improved performance in one comparison (FIG. 7A), in other trials (FIGS. 7B and 7C), the encoded cleavage markers did not perform as well. Thus, the final solution does not include an explicit encoding of a cleavage marker.

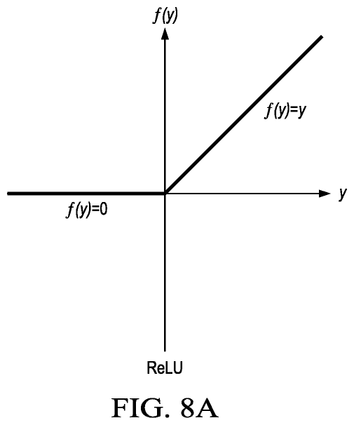

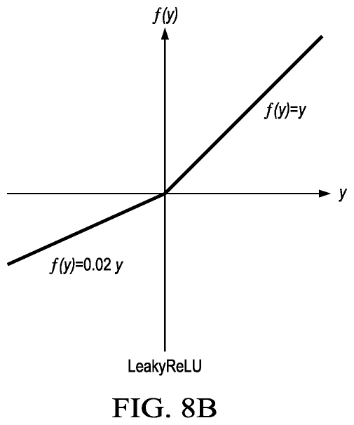

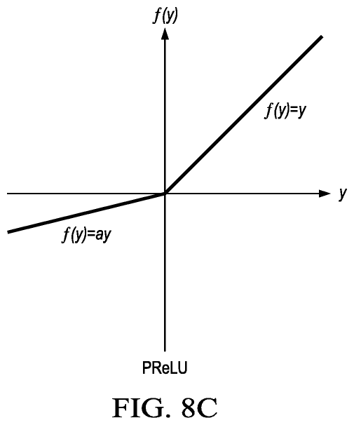

FIG. 8A is a plot of a rectified linear unit (ReLU) activation function according to an exemplary embodiment. FIG. 8B is a plot of a Leaky ReLU activation function according to an exemplary embodiment. FIG. 8C is a plot of a parametric ReLU (PReLU) activation function according to an exemplary embodiment. An activation function may be employed that determines which weights go from one layer to the next. One common type of activation layer is the ReLU, which is used to amplify a weight for the next layer. If anything is negative on the X axis, then ReLU will zero the negative result out; only positive activations are put forward to the next layer.

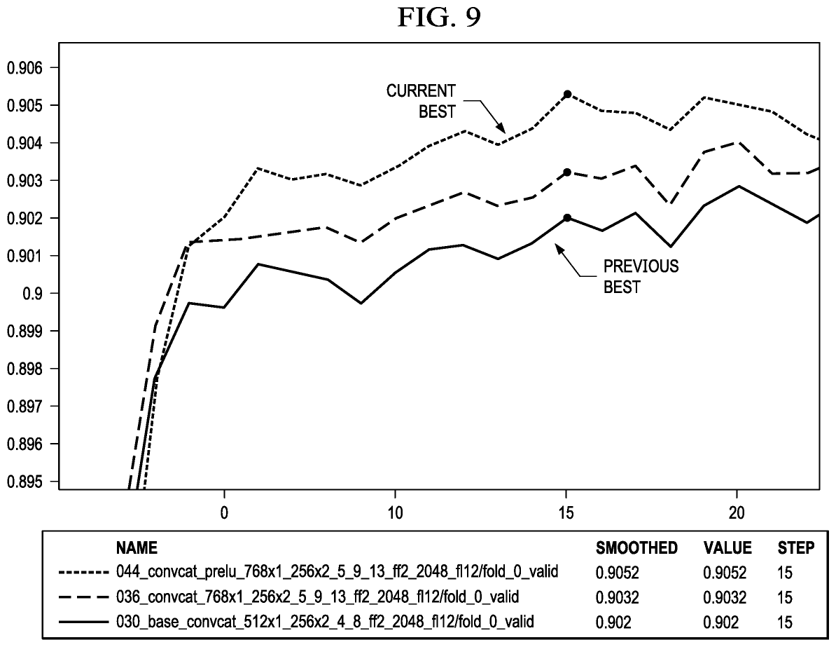

The ReLU was used as an activation function, and then compared to the LeakyReLU, which allows a portion of the negative weights to seep through and to potentially inform the final decision. The LeakyReLU had generally adverse results. Then, PReLU was applied. PReLU generalizes the function. Instead of locking the function to a fixed value, PReLU allows activation layers to be an additional training parameter of the network and allows the network to determine whether there is an optimal value for a given parameter. PReLU proved helpful to the present method. FIG. 9 is a plot of performance of a PReLU model versus two previous top performing models according to an exemplary embodiment. PReLU generated an additional level of improvement.

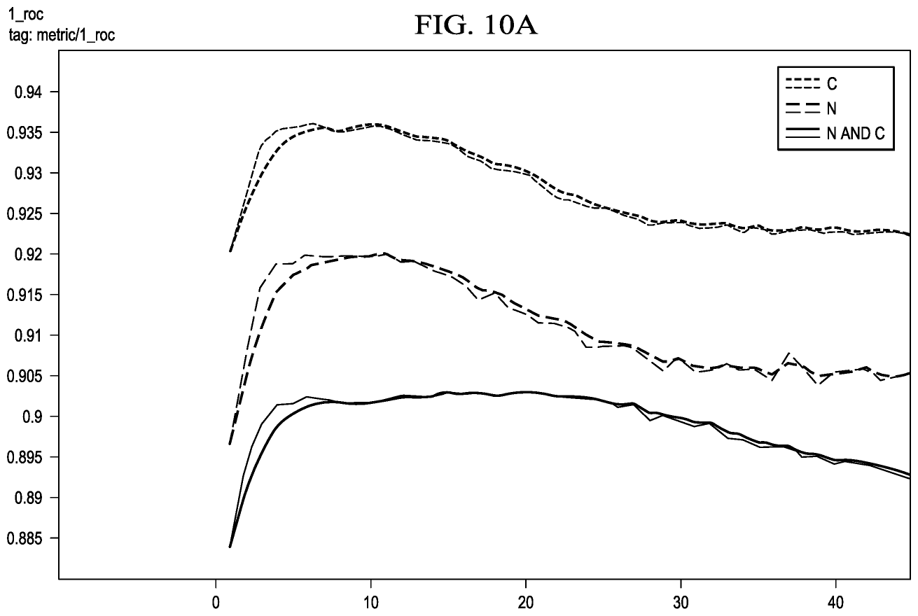

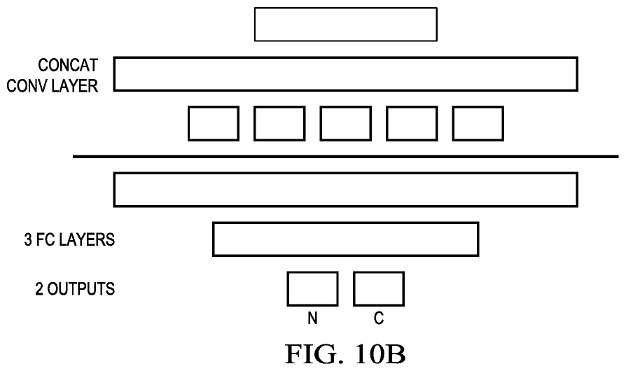

Significant improvement was observed with an architecture having two channel output, i.e., output of a C-terminal cleavage and an N-terminal cleavage. In other words, the model indicates a likelihood of a cleavage to two outputs, one for N-terminal cleavage and one for C-terminal cleavage. FIG. 10A is a plot of performance of a C-terminal, an N-terminal and a combination of the N-terminal and the C-terminal according to an exemplary embodiment. FIG. 10B is a diagram of an architecture of the C-terminal, the N-terminal and the combination of the N-terminal and the C-terminal of FIG. 10A according to an exemplary embodiment. A significant improvement in performance was demonstrated. The initial performance, where the two channels were combined in one and where the difference between the N terminal and the C terminal was not known, was inferior to an output separating the N and C channels.

That is, when the N and C channels are modeled separately, each one of them performs better. As such, when the N- and C-terminals are output separately, the outputs may be more reliably used to generate output peptides, because some ambiguities are eliminated, where otherwise two ambiguous cleavages would be connected that are actually, for example C cleavages. In other words, the separate modeling of the N and C channels was found to be helpful in terms of removing ambiguity of possible cleaved peptides.

By adding the N and C terminals, two different outputs, using the same network except for one of these outputs for two different tasks simultaneously, i.e., if you have two different tasks which in which one is learning the N terminals and the second one is learning the C terminals, two different networks are trained to model probability of cleavage. The model generates data that comprises N terminal cleavages, C terminal cleavages, and decoys. For instance, a most naive scenario involves two networks (e.g., “Net-N” and “Net-C”). In the N network, all the N terminals and all the decoys are trained to predict good N terminal cleavages, and the same for the C network.

However, with the architecture of FIGS. 10A and 10B, by keeping the Concatenated Convolutional Layers, and the three 3 Fully Connected Layers in common, the problem of N terminal and C terminal and only allowing their final outputs to be different, the network is regularized and forced to generalize the problem in a manner that improves performance (e.g., in terms of ROC). The overall system of FIGS. 10A and 10B is much better than either one of these networks or one of these networks separately, because the networks work through the entire set of data overall and are regularizing each other across these tasks.

Table 2 illustrates an exemplary output using an initial cleavage model in which N and C terminals are not modeled (either together or separately).

TABLE 2

| | | |
|---|---|---|
| pos | aa | prob cleavage |

| | | | |
|---|---|---|---|
| 1 | S | 0.002615 | . |
| 2 | D | 0.034558 | . |
| 3 | N | 0.924588 | # |
| 4 | G | 0.007354 | . |
| 5 | P | 0.008825 | . |
| 6 | Q | 0.105593 | . |
| 7 | N | 0.963336 | # |
| 8 | Q | 0.001777 | . |
| 9 | R | 0.002978 | . |
| 10 | N | 0.058732 | . |
| 11 | A | 0.000605 | . |
| 12 | P | 0.075547 | . |
| 13 | R | 0.004033 | . |
| 14 | I | 0.00183 | . |
| 15 | T | 0.002002 | . |
| 16 | F | 0.969697 | # |
| 17 | G | 0.056972 | . |
| 18 | G | 0.005496 | . |
| 19 | P | 0.101563 | . |
| 20 | S | 0.043051 | . |
| 21 | D | 0.202763 | . |
| 22 | S | 0.015211 | . |
| 23 | T | 0.013538 | . |
| 24 | G | 0.01242 | . |
| 25 | S | 0.014121 | . |
| 26 | N | 0.021392 | . |
| 27 | Q | 0.022369 | . |
| 28 | N | 0.328069 | . |
| 29 | G | 0.007329 | . |
| 30 | E | 0.025817 | . |
| 31 | R | 0.715226 | # |
| 32 | S | 0.016519 | . |

In Table 2, “pos” is a position, “aa” is an alphabetic symbol for an amino acid, “prob cleavage” is a probability of cleavage (with 1.0 representing absolute certainty), a dot means a low likelihood of cleavage, and the pound symbol (“#”) indicates a greater than threshold likelihood of cleavage. The initial version showed ambiguity in that it was not clear whether a given cleavage was at the N terminal site or the C terminal site.

Table 3 corresponds, for example, with the model of FIGS. 10A and 10B.

TABLE 3

| | | | |
|---|---|---|---|
| pos | aa | prob cleavage N term | prob cleavage C term |

| | | | | | |
|---|---|---|---|---|---|
| 1 | S | 0.046042 | . | 0 | . |
| 2 | D | 0.034283 | . | 0.000017 | . |
| 3 | N | 0.941907 | N | 0.000008 | . |
| 4 | G | 0.041125 | . | 0 | . |
| 5 | P | 0.034919 | . | 0.00005 | . |
| 6 | Q | 0.054338 | . | 0.000014 | . |
| 7 | N | 0.751842 | N | 0.000004 | . |
| 8 | Q | 0.022662 | . | 0.000001 | . |
| 9 | R | 0.003644 | . | 0.000007 | . |
| 10 | N | 0.468517 | . | 0.000002 | . |
| 11 | A | 0.000014 | . | 0.000014 | . |
| 12 | P | 0.000192 | . | 0.005458 | . |
| 13 | R | 0.00259 | . | 0.005385 | . |
| 14 | I | 0.00011 | . | 0.001987 | . |
| 15 | T | 0.00927 | . | 0.000003 | . |
| 16 | F | 0.000667 | . | 0.970462 | C |
| 17 | G | 0.00322 | . | 0.001265 | . |
| 18 | G | 0.000026 | . | 0.003135 | . |
| 19 | P | 0.005396 | . | 0.117805 | . |
| 20 | S | 0.011935 | . | 0.001041 | . |
| 21 | D | 0.069628 | . | 0.011101 | . |
| 22 | S | 0.001223 | . | 0.002059 | . |
| 23 | T | 0.003744 | . | 0.002439 | . |
| 24 | G | 0.003135 | . | 0.002819 | . |
| 25 | S | 0.007652 | . | 0.021887 | . |
| 26 | N | 0.000937 | . | 0.017219 | . |
| 27 | Q | 0.01263 | . | 0.012787 | . |
| 28 | N | 0.009075 | . | 0.012474 | . |
| 29 | G | 0.010357 | . | 0.00065 | . |
| 30 | E | 0.000967 | . | 0.124297 | . |
| 31 | R | 0.090566 | . | 0.445877 | . |
| 32 | S | 0.002964 | . | 0.108381 | . |

An exemplary output of the improved version is shown in Table 3. The improved model separates the probabilities of cleavage into N-only terminal cleavages and C-only terminal cleavages. In this example, 3 through 16 is one likely peptide, and 7 through 16 is another likely peptide, and so on.

Table 4 depicts a first exemplary higher level interface in which, given a protein P, the model produces a list of possible peptides p_{1 . . . n} in a length range [x . . . y] above a given confidence threshold. In this example, the length range is 9-15, and the probability threshold is at least 0.5000 in one of the two channels.

TABLE 4

| Candidates in range 9-15 | len | N- prob | C- prob |
|---|---|---|---|
| SEQ ID NO: 1 GPQNQRNAPRITF | len 13 | 0.9419 | 0.9705 |
| SEQ ID NO: 2 QRNAPRITF | len 9 | 0.7518 | 0.9705 |
| SEQ ID NO: 3 FPRGQGVPI | len 9 | 0.801 | 0.7947 |
| SEQ ID NO: 4 KMKDLSPRWYFYYL | len 14 | 0.8414 | 0.5068 |
| SEQ ID NO: 5 GQQQQGQTVTK | len 11 | 0.6516 | 0.7625 |
| SEQ ID NO: 6 GQQQQGQTVTKK | len 12 | 0.6516 | 0.8035 |
| SEQ ID NO: 7 RTATKAYNVTQAF | len 13 | 0.6975 | 0.8972 |
| SEQ ID NO: 8 RTATKAYNVTQAFGR | len 15 | 0.6975 | 0.5641 |
| SEQ ID NO: 9 ATKAYNVTQAF | len 11 | 0.537 | 0.8972 |
| SEQ ID NO: 10 ATKAYNVTQAFGR | len 13 | 0.537 | 0.5641 |
| SEQ ID NO: 11 KAYNVTQAF | len 9 | 0.7895 | 0.8972 |
| SEQ ID NO: 12 KAYNVTQAFGR | len 11 | 0.7895 | 0.5641 |
| SEQ ID NO: 13 AQFAPSASAFFGMSR | len 15 | 0.7032 | 0.7338 |
| SEQ ID NO: 14 NFKDQVILLNKHIDA | len 15 | 0.7861 | 0.7484 |
| SEQ ID NO: 15 KTFPPTEPK | len 9 | 0.9392 | 0.7138 |
| SEQ ID NO: 16 KTFPPTEPKK | len 10 | 0.9392 | 0.739 |
| SEQ ID NO: 17 KKADETQALPQRQK | len 14 | 0.6185 | 0.7545 |
| SEQ ID NO: 18 KADETQALPQRQK | len 13 | 0.8715 | 0.7545 |

The length and probabilities may be varied depending on the design specifications for the given protein.

Table 5 depicts a second exemplary higher level interface in which, given a candidate peptide p, and a protein P, the model produces a probability of cleavage and a number of neighboring variants with associated probabilities.

TABLE 5

| candidate | N- prob | C-prob |
|---|---|---|
| GPQNQRNAPRITF | 0.9419 | 0.9705 |
| SEQ ID NO: 1 | | |
| candidate | No prob | C-prob |
| GPQNQRNAPR | 0.9419 | 0.0054 |
| SEQ ID NO: 19 | | |
| candidate | No prob | C-prob |
| LQLPQGTTLPKGF | 0.1887 | 0.0258 |
| SEQ ID NO: 20 | | |

Please note, a neighboring variant may be defined as follows: for any sequence S, sequences S +/−k amino acids (on either the N- or C- terminal) may constitute a neighboring variant within a range of k amino acids. For example, if GPQNQRNAPRITF (SEQ ID NO:1) is the sequence, the variant GPQNQRNAPR (SEQ ID NO:19) is the sequence minus three amino acids from the C-terminal. Also, conversely, if GPQNQRNAPR (SEQ ID NO:19) is the sequence, the variant GPQNQRNAPRITF (SEQ ID NO:1) is the sequence plus three amino acids at the C-terminal. The protein input to the system may be a fixed string of some length L, and the system may be configured to explore various contiguous substrings of that protein.

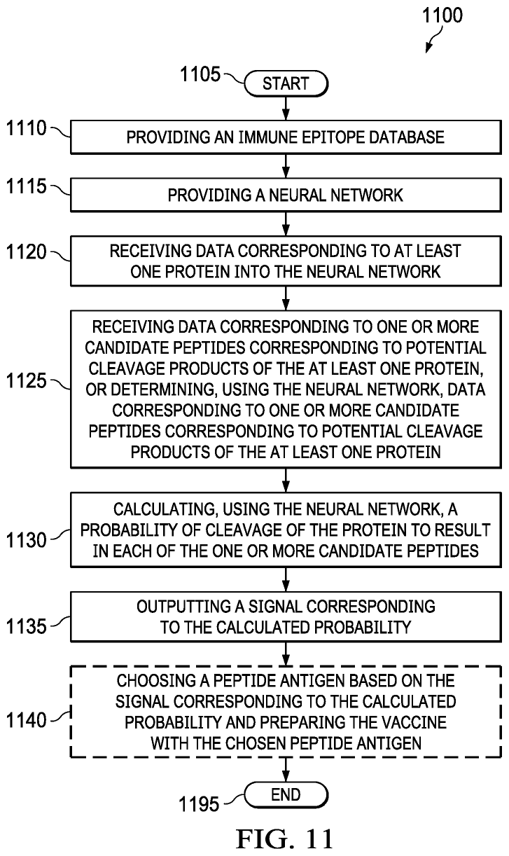

FIG. 11 is a flow chart of a method for protein analysis according to an exemplary embodiment. The method 1100 may include a start 1105 and an end 1195. The method 1100 may include providing an immune epitope database (1110). The method 1100 may include providing a neural network (1115). The method 1100 may include receiving data corresponding to at least one protein into the neural network (1120). The method 1100 may include receiving data corresponding to one or more candidate peptides corresponding to potential cleavage products of the at least one protein, or determining, using the neural network, data corresponding to one or more candidate peptides corresponding to potential cleavage products of the at least one protein (1125). The method 1100 may include calculating, using the neural network, a probability of cleavage of the protein to result in each of the one or more candidate peptides (1130). The method 1100 may include outputting a signal corresponding to the calculated probability (1135). The method 1100 may include choosing a peptide antigen based on the signal corresponding to the calculated probability and preparing the vaccine with the chosen peptide antigen (1140).

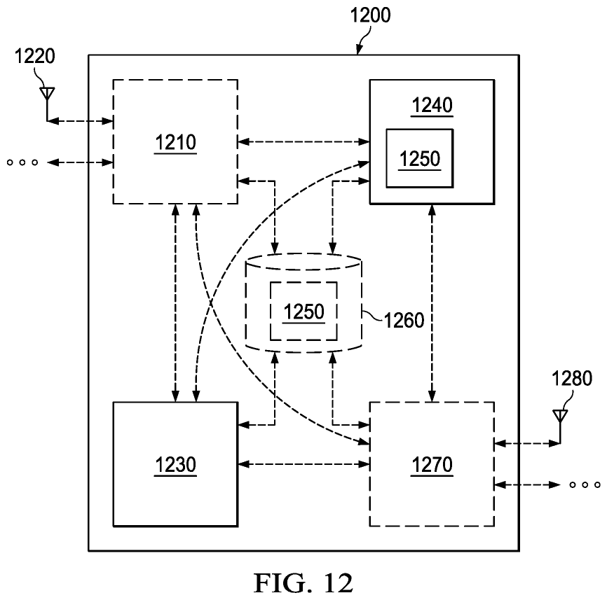

FIG. 12 is a schematic diagram of a computer device or system including at least one processor and a memory storing at least one program for execution by the at least one processor according to an exemplary embodiment. Specifically, FIG. 12 depicts a computer device or system 1200 comprising at least one processor 1230 and a memory 1240 storing at least one program 1250 for execution by the at least one processor 1230. In some embodiments, the device or computer system 1200 can further comprise a non-transitory computer-readable storage medium 1260 storing the at least one program 1250 for execution by the at least one processor 1230 of the device or computer system 1200. In some embodiments, the device or computer system 1200 can further comprise at least one input device 1210, which can be configured to send or receive information to or from any one from the group consisting of: an external device (not shown), the at least one processor 1230, the memory 1240, the non-transitory computer-readable storage medium 1260, and at least one output device 1270. The at least one input device 1210 can be configured to wirelessly send or receive information to or from the external device via a means for wireless communication, such as an antenna 1220, a transceiver (not shown) or the like. In some embodiments, the device or computer system 1200 can further comprise at least one output device 1270, which can be configured to send or receive information to or from any one from the group consisting of: an external device (not shown), the at least one input device 1210, the at least one processor 1230, the memory 1240, and the non-transitory computer-readable storage medium 1260. The at least one output device 1270 can be configured to wirelessly send or receive information to or from the external device via a means for wireless communication, such as an antenna 1280, a transceiver (not shown) or the like.

Each of the above identified modules or programs corresponds to a set of instructions for performing a function described above. These modules and programs (i.e., sets of instructions) need not be implemented as separate software programs, procedures or modules, and thus various subsets of these modules may be combined or otherwise re-arranged in various embodiments. In some embodiments, memory may store a subset of the modules and data structures identified above. Furthermore, memory may store additional modules and data structures not described above.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described herein can include electrical circuit(s) that can include components and circuitry elements of suitable value in order to implement the embodiments of the subject innovation(s). Furthermore, it can be appreciated that many of the various components can be implemented on at least one integrated circuit (IC) chip. For example, in one embodiment, a set of components can be implemented in a single IC chip. In other embodiments, at least one of respective components are fabricated or implemented on separate IC chips.

What has been described above includes examples of the embodiments of the present invention. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that at least one component may be combined into a single component providing aggregate functionality or divided into several separate sub-components, and any at least one middle layer, such as a management layer, may be provided to communicatively couple to such sub-components in order to provide integrated functionality. Any components described herein may also interact with at least one other component not specifically described herein but known by those of skill in the art.

In addition, while a particular feature of the subject innovation may have been disclosed with respect to only one of several implementations, such feature may be combined with at least one other feature of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

As used in this application, the terms “component,” “module,” “system,” or the like are generally intended to refer to a computer-related entity, either hardware (e.g., a circuit), a combination of hardware and software, software, or an entity related to an operational machine with at least one specific functionality. For example, a component may be, but is not limited to being, a process running on a processor (e.g., digital signal processor), a processor, an object, an executable, a thread of execution, a program, and/or a computer. By way of illustration, both an application running on a controller and the controller can be a component. At least one component may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

Moreover, the words “example” or “exemplary” are used herein to mean serving as an example, instance, or illustration. Any aspect or design described herein as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Computing devices typically include a variety of media, which can include computer-readable storage media and/or communications media, in which these two terms are used herein differently from one another as follows. Computer-readable storage media can be any available storage media that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by at least one local or remote computing device, e.g., via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g., a carrier wave or other transport mechanism, and includes any information delivery or transport media. The term “modulated data signal” or signals refers to a signal that has at least one of its characteristics set or changed in such a manner as to encode information in at least one signal. By way of example, and not limitation, communication media include wired media, such as a wired network or direct-wired connection, and wireless media such as acoustic, RF, infrared and other wireless media.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described herein. Furthermore, not all illustrated acts may be required to implement the methodologies in accordance with the disclosed subject matter. In addition, those skilled in the art will understand and appreciate that the methodologies could alternatively be represented as a series of interrelated states via a state diagram or events. Additionally, it should be appreciated that the methodologies disclosed in this specification are capable of being stored on an article of manufacture to facilitate transporting and transferring such methodologies to computing devices. The term article of manufacture, as used herein, is intended to encompass a computer program accessible from any computer-readable device or storage media.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Although at least one exemplary embodiment is described as using a plurality of units to perform the exemplary process, it is understood that the exemplary processes may also be performed by one or plurality of modules.

The use of the terms “first”, “second”, “third” and so on, herein, are provided to identify various structures, dimensions or operations, without describing any order, and the structures, dimensions or operations may be executed in a different order from the stated order unless a specific order is definitely specified in the context.

Approximating language, as used herein throughout the specification and claims, may be applied to modify any quantitative representation that could permissibly vary without resulting in a change in the basic function to which it is related. Accordingly, a value modified by a term or terms, such as “about” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

### Methods of Use

The systems, apparatus, methods, and/or articles described herein may be used in any scenario where prediction of protein cleavage by human cells, and/or presentation of those peptides by WIC, is desired.

In some aspects is provided a method for preparing a vaccine. In some embodiments, the vaccine is formulated for administration to a patient. In some embodiments, the vaccine is a preventative vaccine. In some embodiments, the vaccine is a treatment vaccine.

In some embodiments, the method includes combining one or more peptides with a pharmaceutically acceptable excipient. In some embodiments, the one or more peptides are determined to be likely cleavage products from a protein of interest. In some embodiments, the protein of interest is associated with a disease, e.g., a tumor-associated antigen or neoantigen. In some embodiments, the protein of interest is specific to a disease, e.g., a tumor-specific antigen or neoantigen, or a viral protein. In some embodiments, the disease is a cancer. In some embodiments, the disease is an infection. In some embodiments, the protein is associated with a disease in a particular patient, i.e., the vaccine is a bespoke vaccine. In some embodiments, the protein is associated with a disease in a subset of patients.

In some embodiments, the vaccine is to be administered to a patient in need thereof. In some embodiments, a biological sample is obtained from the patient. In some embodiments, the biological sample is analyzed to determine one or more disease-associated or -specific proteins. The one or more disease-associated or -specific proteins may be analyzed by the methods, systems, and/or apparatus as described herein. The resulting peptide(s) may be used to prepare a vaccine. The vaccine may be administered to the patient.

In some embodiments, the methods, systems, and/or apparatus as described herein may be used to determine peptides for use in a vaccine for prevention of a disease. In some embodiments, the peptides are from a protein associated with that disease. In some embodiments, the disease is an infectious disease. In some embodiments, the disease is a cancer. In some embodiments, the vaccine is administered to a healthy individual.

In some embodiments, the methods, systems, and/or apparatus as described herein may be used to determine peptides for use in a vaccine for treatment of a disease. In some embodiments, the peptides are from a protein associated with that disease. In some embodiments, the peptides are from more than one protein associated with that disease. In some embodiments, biological samples are provided from multiple patients having the disease, or a database of proteins associated with the disease is utilized to determine proteins of interest. In some embodiments, the biological sample or database is analyzed to determine one or more disease-associated or -specific proteins. The one or more disease-associated or -specific proteins may be analyzed by the methods, systems, and/or apparatus as described herein. The resulting peptide(s) may be used to prepare a vaccine. In some embodiments, the disease is an infectious disease. In some embodiments, the disease is a cancer.

In some embodiments, the vaccine is administered to a patient in need thereof

The subject matter described herein may be embodied in systems, apparatus, methods, and/or articles depending on the desired configuration. The embodiments set forth in the foregoing description do not represent all embodiments consistent with the subject matter described herein. Instead, they are merely some examples consistent with aspects related to the described subject matter. Although a few variations have been described in detail above, other modifications or additions are possible. In particular, further features and/or variations may be provided in addition to those set forth herein. For example, the embodiments described above may be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other embodiments may be within the scope of the following claims.

1. A method of preparing a vaccine comprising a peptide antigen or an immunotherapy treatment for cancer comprising a peptide antigen, wherein a device includes at least one processor and a memory storing at least one program for execution by the at least one processor, the at least one program including instructions which, when executed by the at least one processor, cause the at least one processor to perform the method, the method comprising:
providing an immune epitope database;
providing a neural network;
receiving data corresponding to at least one protein into the neural network;
receiving data corresponding to one or more candidate peptides corresponding to potential cleavage products of the at least one protein, or determining, using the neural network, data corresponding to one or more candidate peptides corresponding to potential cleavage products of the at least one protein;
calculating, using the neural network, a probability of cleavage of the protein to result in each of the one or more candidate peptides; and
outputting a signal corresponding to the calculated probability.

2. The method of claim 1, further comprising choosing a peptide antigen based on the signal corresponding to the calculated probability and preparing the vaccine with the chosen peptide antigen.

3. The method of claim 2, wherein the choosing the peptide antigen based on the signal corresponding to the calculated probability is based on a determination of whether the calculated probability is within a predetermined range of values.

4. The method of claim 1, wherein the calculating, using the neural network, the probability of cleavage for each of the one or more candidate peptides includes:
calculating, using the neural network, a probability of cleavage for at least one N-terminal of each of the one or more candidate peptides; or
calculating, using the neural network, a probability of cleavage for at least one C-terminal of each of the one or more candidate peptides.

5. The method of claim 1, wherein the calculating, using the neural network, the probability of cleavage for each of the one or more candidate peptides includes:
calculating, using the neural network, a probability of cleavage for at least one N-terminal of each of the one or more candidate peptides; and
independent of the N-terminal calculation, calculating, using the neural network, a probability of cleavage for at least one C-terminal of each of the one or more candidate peptides.

6. The method of claim 1, further comprising:
determining, using the neural network, data corresponding to one or more neighboring variants of the one or more candidate peptides; and
calculating a probability of cleavage for the one or more neighboring variants.

7. The method of claim 1, wherein the immune epitope database includes data representing one or more unique antigen proteins, one or more unique peptides, one or more unique peptide/protein pairs, and one or more decoys.

8. The method of claim 1, wherein the immune epitope database is restricted to major histocompatibility complex (MHC) pathways, MHC Class I (MHC-I) pathways, human-only immune epitopes, or sequences that positively bind to MHC.

9-11. (canceled)

12. The method of claim 1, wherein the immune epitope database includes tandem mass spectrometry data where a single MHC-allele is not identified.

13. The method of claim 1, wherein a flank size for each of the one or more candidate peptides is greater than or equal to 6 and less than or equal to 20.

14. (canceled)

15. The method of claim 1, wherein a measurement of an accuracy of the calculating, using the neural network, the probability of cleavage for each of the one or more candidate peptides includes a receiver operating characteristic (ROC), and wherein an ROC closest to 1.0 is ideal.

16. The method of claim 1, wherein the neural network includes:
one or more convolutional layers; and
one or more fully connected layers, and
wherein the one or more convolutional layers consists of a single convolutional layer.

17. The method of claim 1, wherein the neural network includes:
one or more convolutional layers; and
one or more fully connected layers,
wherein the one or more convolutional layers comprises the one or more convolutional layers in parallel, and
wherein each of the one or more convolutional layers has a different size kernel.

18. The method of claim 1, wherein one or more candidate peptides are modeled without an explicit encoding of a cleavage marker.

19. The method of claim 1, wherein the neural network includes a parametric rectified linear unit activation function.

20. The method of claim 1, wherein the outputting the signal corresponding to the calculated probability includes one or more of the following:
generation of a first table including a position column, an antigen marker, a probability of cleavage, and an indicator of cleavage or a pad;
generation of a second table including data for an N-terminal and data for a C-terminal, wherein each of the data for the N-terminal and the data for the C-terminal includes: a position column, an antigen marker, an N-terminal probability of cleavage, an N-terminal indicator of cleavage or a pad; a C-terminal probability of cleavage, and a C-terminal indicator of cleavage or a pad;
generation of a third table including a candidate peptide column, a length column, an N-terminal probability, and a C-terminal probability; and
generation of a fourth table including a candidate peptide column, an N-terminal probability, and a C-terminal probability, wherein the candidate peptide column includes one or more neighboring variants of the one or more candidate peptides.

21. The method of claim 1, wherein the vaccine is for an infectious disease or a cancer.

22. (canceled)

23. The method of claim 21, wherein the at least one protein is a tumor-associated antigen, a neoantigen, or an antigen from a virus, bacterium, fungus, protozoa, prion, or helminth.

24-25. (canceled)

26. A system for preparing a vaccine comprising a peptide antigen or an immunotherapy treatment for cancer comprising a peptide antigen, the system comprising:
a device having at least one processor and a memory storing at least one program for execution by the at least one processor, wherein the at least one program includes instructions which, when executed by the at least one processor, cause the at least one processor to perform operations comprising:
providing an immune epitope database;
providing a neural network;
receiving data corresponding to at least one protein into the neural network;
receiving data corresponding to one or more candidate peptides corresponding to potential cleavage products of the at least one protein, or determining, using the neural network, data corresponding to one or more candidate peptides corresponding to potential cleavage products of the at least one protein;
calculating, using the neural network, a probability of cleavage of the protein to result in each of the one or more candidate peptides; and
outputting a signal corresponding to the calculated probability.

27-50. (cancelled)

51. A non-transitory computer-readable storage medium storing at least one program for preparing a vaccine comprising a peptide antigen or an immunotherapy treatment for cancer comprising a peptide antigen, the at least one program configured for execution by at least one processor and a memory storing the at least one program, the at least one program including instructions which, when executed by the at least one processor, cause the at least one processor to perform operations comprising:
providing an immune epitope database;
providing a neural network;
receiving data corresponding to at least one protein into the neural network;
receiving data corresponding to one or more candidate peptides corresponding to potential cleavage products of the at least one protein, or determining, using the neural network, data corresponding to one or more candidate peptides corresponding to potential cleavage products of the at least one protein;
calculating, using the neural network, a probability of cleavage of the protein to result in each of the one or more candidate peptides; and
outputting a signal corresponding to the calculated probability.

52-81. (canceled)
